(19) United States
(12) Plant Patent Application Publication
(10) Pub. No.: US 2013/0019364 P1
(43) Pub. Date: Jan. 17, 2013

(54) Hosta plant named 'Forbidden Fruit'

(50) ;

(71) Applicant: Marco Fransen, Tel Aar (NL)

(72) Inventor: Marco Fransen, Tel Aar (NL)

(21) Appl. No.: 13/135,870

(22) Filed: Jul. 14, 2011

(51) Int. Cl. A01H 5/00

(52) U.S. Cl. PLT/353

(57) ABSTRACT

A new and distinct Hosta cultivar named ‘Forbidden Fruit’ is disclosed, characterized by distinctive large, bright foliage with a green margin, vigorous plant habit and strong foliage, with good resistance to environmental stressors and large flowers. The near variety is a Hosta, typically used for outdoor ornamental purposes.

### LATIN NAME OF THE GENUS AND SPECIES

Hosta hybrid

### VARIETY DENOMINATION

‘FORBIDDEN FRUIT’

### BACKGROUND OF THE INVENTION

The new cultivar was discovered as an induced mutation using the known chemical colchicine, of Hosta hybrid ‘Orange Marmalade’ U.S. Plant Pat. No. 16,742 ‘Forbidden Fruit’ was discovered by the inventor, Marco Fransen, a citizen of the Netherlands in October of 2008, at a research facility in Ter Aar, The Netherlands. The intent of the breeding program was to create new tetraploid Hosta hybrids with strong foliage and forming vigorous plants.

Directly after discovery of the tetraploid mutation, the inventor initiated cultivation in tissue culture, during October of 2008, in a commercial laboratory in Iribov Heerhugowaard, the Netherlands. Subsequently ‘Forbidden Fruit’ has been reproduced by micro-propagation and has shown that the unique features of this cultivar are stable and reproduced true to type through several generations.

### SUMMARY OF THE INVENTION

The cultivar ‘Forbidden Fruit’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Forbidden Fruit’ These characteristics in combination distinguish ‘Forbidden Fruit’ as a new and distinct Hosta cultivar:

1. Distinctive, bright foliage with a broad green margin.
2. Thick foliage resistant to environmental stressors.
3. Large foliage.
4. Vigorous plant.
5. Large flowers.

### COMPARISON TO PARENT

Plants of the new cultivar ‘Forbidden Fruit’ are similar to plants of the parent; Hosta hybrid ‘Orange Marmalade’ in most horticultural characteristics, however, plants of the new cultivar ‘Forbidden Fruit’ are more vigorous and produce thicker, stronger leaves and thicker petioles than the parent variety. Additionally, the new variety produces larger foliage than ‘Orange Marmalade’. Leaves of ‘Forbidden Fruit’ have a wider green margin than ‘Orange Marmalade’.

### COMMERCIAL COMPARISON

The most similar commercial Hosta hybrid variety available to compare to ‘Forbidden Fruit’ is the parent variety.

### BRIEF DESCRIPTION OF THE PHOTOGRAPH

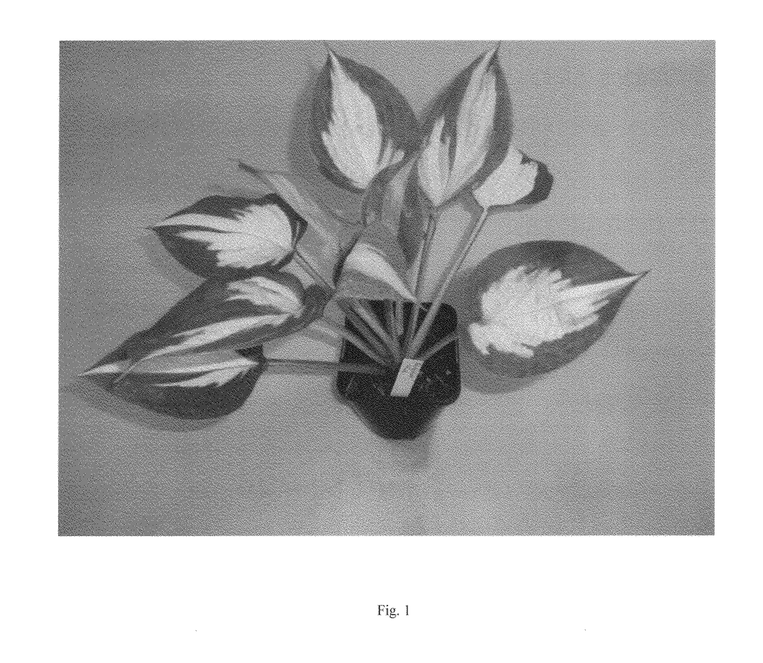

The accompanying photograph in FIG. 1 illustrates in full color a typical plant of ‘Forbidden Fruit’ grown outdoors. This plant is approximately 6 months old, shown in a 1 liter pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart, 2001, except where general terms of ordinary dictionary significance are used. The following observations and measurements describe ‘Forbidden Fruit’ plants grown in a commercial nursery in the Netherlands, from February of 2011 through June of 2011. Temperatures were 5° C. to 20° C. at night and 10° C. to 30° C. during the day. Measurements and numerical values represent averages of typical plant types. No growth regulators or special treatments were used.
Botanical classification: Hosta hybrid ‘Forbidden Fruit’.
Propagation:
   Time to initiate roots.—Approximately 14 to 21 at 23° C.
   Root description.—Fleshy with moderate side roots.
Plant:
   Growth habit.—Outwardly arching herbaceous perennial plant.
   Height.—Approximately 35 cm in a 1 liter pot.
   Plant spread.—Approximately 45 cm.
   Growth rate.—Moderate.
   Branching characteristics.—Branches emerge directly from the roots.
   Age of plant described.—Approximately 4 months from a stage 3 tissue culture.
Foliage:
   Leaf.—Quantity per plant: Average 20 Leaf Type: Simple, basal emerging Average Leaf Length: Approximately 35 cm Average Leaf Width: Approximately 18 cm, at widest point Apex: Acute Base: Cordate. Overall Leaf Shape: Obovate Overall Leaf Aspect: Arching Margin: Slightly undulating Texture of top surface: Smooth Texture of bottom surface: Smooth Appearance of top surface: Slightly leathery. Appearance of bottom surface: Slightly leathery. Pubescence: No pubescence on leaves Color: Young foliage upper side: Near RHS Yellow-Green 150B, center Green-Yellow 1C margin near RHS Green 137A Young foliage under side: Near RHS Green-Yellow 1B, margin near RHS Green 137B. Mature foliage upper side: Near RHS Yellow 4B, center Green-Yellow 1D margin near RHS Green 137A Mature foliage under side: Near RHS Yellow 4B, center Green-Yellow 1D margin near RHS Green 137A Venation: Type: Pinnate. Venation coloration upper side: Indistinguishable from foliage color. Venation coloration under side: Indistinguishable from foliage color.
   Leaf petiole.—Length: Approximately 12 cm Width: Approximately 2.1 cm at widest point Color: Upper side: Near RHS Green-Yellow 1B Under side: Near RHS Yellow-Green N144C Strength: Very strong.
   Other foliage characteristics.—Thick foliage, resistant to mechanical damage and environmental stressors.
Flower:
   Natural flowering season.—Late Spring into Summer.
   Inflorescence type and habit.—Typically — Single, campanulate arranged in racemes.
   Rate of flower opening.—Lowest flower opens first, all flowers have opened in approx. 2 weeks.
   Flower longevity on plant.—Approx. 2 weeks.
   Quantity of flowers.—Average: 13 flowers per raceme.
   Raceme size.—Height: Approximately 14.0 cm, from lowest flower to top of Inflorescence. Width: Approximately 11.3 cm.
   Flower.—Perianth: Arrangement: Campanulate Overall FlowerSize: Length: Approximately 6.7 cm Width: Approximately 2.7 cm at widest point.
   Color.—Tepals: When opening: Outer surface:Near RHS White NN155D, slightly tinged Purple 76C Inner surface:Near RHS White NN155D, slightly tinged Purple 76C Fully opened: Outer surface: Near RHS NN155D Inner surface:Near RHS NN155D Color Changes when Aging: Near RHS NN155D.
   Sepals or bracts.—Quantity: One bract is placed under each individual flower Shape: Ovate Length: Approximately 1.9 cm. Diameter: Approximately 0.8 cm Color: Outer surface: Near RHS N144D Inner surface: Near RHS N144D Color Changes when Aging: Near RHS Greyed-Orange 164C.
   Bud.—Shape: Obovate. Length: Approximately 3.3 cm. Diameter: Approximately 1.0 cm. Color: Near RHS NN155D, slightly tinged 76C, base near RHS Yellow-Green 145B.
   Pedicels.—Length: Approximately 0.7 cm. Width: Approximately 0.2 cm. Aspect: Placed to the petiole in an average angle of 30° (0° =horizontal), curved downward in an average angle of 30° (0°=horizontal). Color: Near RHS Yellow-Green 145C.
   Scape.—Length: Approximately 50.6 cm Width: Approximately 0.4 cm Angle: Near vertical Strength: Strong Color: Near RHS Yellow-Green 145A.
   Fragrance.—No.
Reproductive organs:
   Stamens.—Number: Average 6 Filament Length: Approximately 4.9 cm. Filament Color: Near RHS Yellow-Green 145D Anther length: Approximately 0.6 cm Anther color: Near RHS Greyed-Orangel76B Anther shape: Oblong, dorsifixed Pollen: Moderate quantity, colored near RHS Yellow-Orange 17B.
   Pistil.—Number: 1 Length: Approximately 6.3 cm. Style color: Near RHS Green-White 157C Stigma: Shape: Club-shaped Color: Near RHS N155D Ovary color: Near RHS 145A.
Other characteristics:
Disease resistance: Better resistance to diseases or pests has been observed in this variety, due to the strong, thick foliage.
Temperature rolerance: Hardy perennial, tolerates a temperature range from approximately −30° C. to at least 40° C.
Drought rolerance: No tolerance for drought.

What is claimed is:

1. A new and distinct cultivar of Hosta plant named ‘Forbidden Fruit’ as herein illustrated and described.
